(19) United States
(12) Patent Application Publication
(10) Pub. No.: US 2011/0054296 A1
(43) Pub. Date: Mar. 3, 2011

(54) REMOTE DISPLAY FOR MEDICAL SCANNING APPARATUS

(75) Inventors: Matthew McCarthy, Thebarton (AU); Stewart Bartlett, Thebarton (AU)

(73) Assignee: SIGNOSTICS LIMITED, Torrensville, SA (AU)

(21) Appl. No.: 12/866,458

(22) PCT Filed: Feb. 6, 2009
(86) PCT No.: PCT/AU2009/000130

§ 371 Date: Aug. 6, 2010

(30) Foreign Application Priority Data
Feb. 7, 2008 (AU) 2008900562

(51) Int. Cl. A61B 5/05

(52) U.S. Cl. 600/407

(57) ABSTRACT

A hand held medical diagnostic apparatus including a processing unit and a probe adapted to produce medical diagnostic data. The processing unit receives medical diagnostic data and processes It to produce display Information. There is a communications channel adapted to transmit data between the processing unit and a remote display unit located remotely from the probe and from the processing unit. The processing unit is adapted to send the display information via the communications channel to the remote display unit and the remote display unit is adapted to display the display information on a screen.

### TECHNICAL FIELD

The present invention relates to a remote display for a medical scanning apparatus, in particular a hand-held medical diagnostic apparatus.

### BACKGROUND ART

Medical and veterinary practitioners often need to perform numerous tests and procedures on a patient to diagnose illness. The diagnosis of illness usually involves several stages. The first stage is a series of questions and simple diagnostic tests. This stage is relatively inexpensive to perform, and is performed at the patient bedside or in a general/family practice office, if the physician suspects a problem, is unsure, or needs further information, a second stage of test is performed which could include ultrasound imaging, magnetic resonance imaging (MRI), X-Ray, or Computer Aided Tomography. These tests are more expensive, but are still non-invasive. A third stage of tests can be performed including using catheters to inject imaging substances into a patient for clearer images (eg X-Ray, MRI, CAT, Ultrasound). A fourth stage would be exploratory surgery.

The accuracy and ability of physicians in the first stage of testing has a significant impact on the overall efficiency of a health system. Unnecessary referral for further tests results in waste and unnecessary expense. The first stage of diagnoses includes but is not limited to auscultation, pulse detection, ear and eye inspection, blood pressure detection, visual inspection, temperature detection, neurological tests, and percussion. These tests are carried out using either separate devices or with fingers, hands, eyes, and ears. Some diagnoses require a detailed process of individual tests with the combination of results providing disease indicators.

Devices a physician uses during preliminary examination include stethoscopes, otoscopes, ophthalmoscopes, thermometers, pressure detectors, and neurological kits. Other procedures include palpating to detect arterial pulses, glucose testing, percussing (tapping and listening to the characteristics of the sound) and palpation to detect sub-dermal structure, and visual inspection for examining jugular venous pressure and characteristics.

All of these devices, when portable, must be carried and stored individually. Many now include electronic or electrical features and these then require battery power and generally separate battery chargers for each device. When the devices are not portable, or not easily carried, the difficulty of bringing them to the patient may lead to such devices not being used in the first instance, contributing to unnecessary further testing.

However, portability of a diagnostic device inherently reduces the size and useful resolution of a display screen which is part of the device.

This limits the usefulness of the device in some specific circumstances, and may make training users in the use of the device more difficult.

Other objects and advantages of the present invention will become apparent from the following description, taken in connection with the accompanying drawings, wherein, by way of illustration and example, an embodiment of the present invention is disclosed.

### DISCLOSURE OF THE INVENTION

In one form of this invention although this may not necessarily be the only or indeed the broadest form of this there is proposed a hand held medical diagnostic apparatus including a processing unit including a probe adapted to produce medical diagnostic data;

the processing unit being adapted to receive said medical diagnostic data and to process it to produce display information for display to a user. There is a communications channel adapted to transmit data between the processing unit and a remote display unit located remotely from the probe and from the processing unit. The processing unit is adapted to send the display information via the communications channel to the remote display unit and the remote display unit is adapted to display the display information on a screen.

In preference, the probe unit and the processing unit are located in a common housing.

In the alternative, the hand held medical diagnostic apparatus of claim 1 wherein the probe unit and the processing unit are located in separate housings.

The remote display unit includes general purpose computing functionality, adapted to display the display information from the processing unit, for example a personal computer, a PDA or a mobile phone with processing capability which may be termed a smartphone.

The processing unit housing will usually include a local display screen. The processing unit will be adapted to display diagnostic information or the results of diagnostic tests or scans on the local display screen.

In general the display specifications of the remote display unit screen are significantly different from the display specifications of the local display screen and the display information displayed on each of the screens by the processing unit in response to the medical diagnostic data is substantially different.

There are situations when the display screen included in the processing unit housing, may be considered to be too small.

This may be because it is desirable for more than one person to be able to see the screen at one time. In a clinical setting where the opinion of other practitioners is to be sought, it is advantageous to be able to have several people view the display simultaneously.

In teaching situations, it is useful for alt participants to be able to see the displayed information at the same time. A larger, remote display allows larger groups to be trained in the use of the device, whilst all trainees are able to see the display information simultaneously.

The larger remote display unit is also advantageous in a clinical situation when a clearer view of the information being displayed is required, in order to see smaller features or finer detail.

### BRIEF DESCRIPTION OF THE DRAWINGS

FIG. 1 illustrates a preferred embodiment of a medical diagnostic device of the invention.

FIG. 2 is a block diagram of the medical diagnostic device of FIG. 1, Incorporating an embodiment of the invention.

FIG. 3 is a functional block diagram of a remote display unit of the embodiment of FIG. 2.

FIG. 4 is a block diagram of the medical diagnostic device of FIG. 1, incorporating a further embodiment of the invention.

FIG. 5 is a functional block diagram of a remote display unit of the embodiment of FIG. 4.

FIG. 6 illustrates an alternative embodiment of the invention without a local display.

FIG. 7 is a block diagram of an embodiment of the invention, wherein the medical diagnostic function is that of an ultrasound scanner.

### BEST MODE FOR CARRYING OUT THE INVENTION

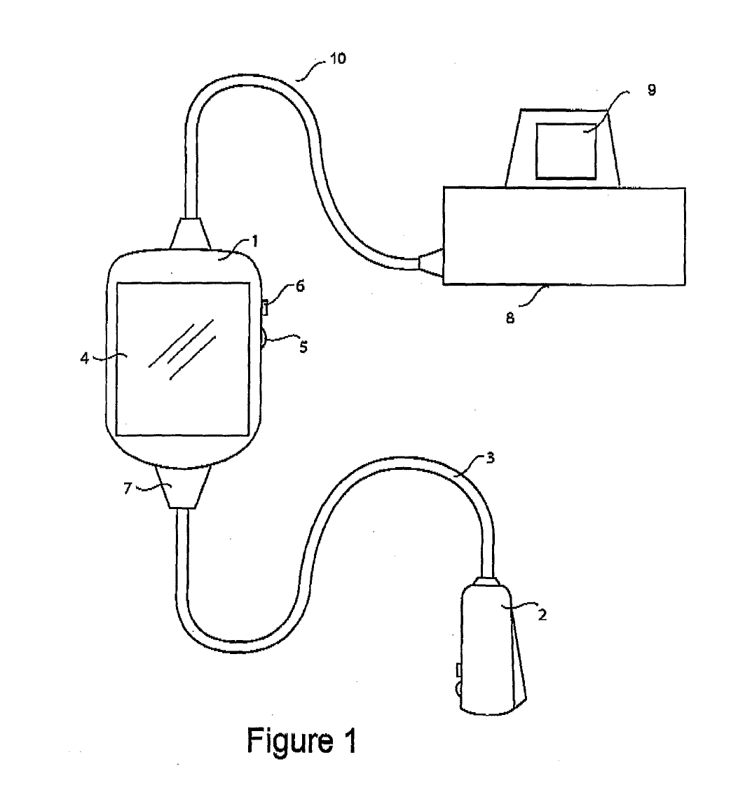

Referring to FIG. 1 there is illustrated a portable diagnostic device to be used by physicians at the bedside. There is a handheld display and processing unit (DPU) 1 connected to a diagnostic probe unit 2 via a cable 3. The cable attaches to the DPU via a plug and socket arrangement 7. In other embodiments, the plug and socket may be at the probe unit end of the cable, or may be provided at each end of the cable. The cable may also be permanently attached to both probe unit and DPU, in which case only the functionality of the attached probe unit is available.

A variety of diagnostic probe units incorporating different types of sensors providing one or more diagnostic functions can be attached to the DPU. The DPU provides a configurable (programmable) Interface, where the interface configuration is provided by the probe unit upon connection. The DPU does not need any user intervention to identify the requirements of a probe unit when it is plugged into the DPU. The interface provides a configurable data interface and may also supply power and an optical input interface.

The handheld display and processing unit 1 and diagnostic probe unit 2 are designed to be of substantially equivalent mass, enabling the system to be conveniently stored around a users neck, enhancing the portability of the device.

The diagnostic probe adapts the system to any suitable diagnostic function. This function may be without limitation, that of audio devices, ultrasound scanners, otoscopes, ophthalmoscopes, blood testing devices, endoscopes, electro cardiogram devices, skin lesion testing devices, and vital signs testing devices.

The DPU includes a miniature colour display 4, which is a 320×200 pixel LCD display in this embodiment. Any display which is small enough to fit into the DPU may be used.

A variety of user input apparatus are provided. The handheld display and processing unit 1 provides a scroll wheel 5 and a pushbutton 6 for user input to allow control of most operations.

In further embodiments, the screen 4 may be a touch sensitive screen, allowing user input with or without a stylus. A Bluetooth interface may be provided enabling the use of wireless keyboards or input devices. A microphone in conjunction with a dictation processing application may be provided for use for voice recording.

Upon connection of any one of the probe units, the DPU automatically reconfigures the interface to the probe unit to provide the required communication protocol for communication with the probe unit, and runs software to provide the appropriate display and control features for the functionality of the connected probe unit.

There is also provided a remote display unit 8, which in this embodiment is a personal computer. The remote display unit 8 includes a remote display unit screen 9, which is in general larger and/or of a higher resolution than the display screen 4 on the DPU 1. The remote display unit is linked to the DPU 1 via a communication channel 10.

There are situations when the display screen 4 on the DPU 1, may be considered to be too small.

This may be because it is desirable for more than one person to be able to see the screen at one time. In a clinical setting where the opinion of other practitioners is to be sought, it is advantageous to be able to have several people view the display simultaneously.

In teaching situations, it is useful for all participants to be able to see the displayed information at the same time. A larger, remote display allows larger groups to be trained in the use of the device, whilst all trainees are able to see the display information simultaneously.

The larger remote display unit is also advantageous in a clinical situation when a dearer view of the information being displayed is required, in order to see smaller features or finer detail.

The remote display unit is a device with general purpose processing functionality. It may be a personal computer, PDA, a mobile phone or any other device including functionality for executing user supplied computer code.

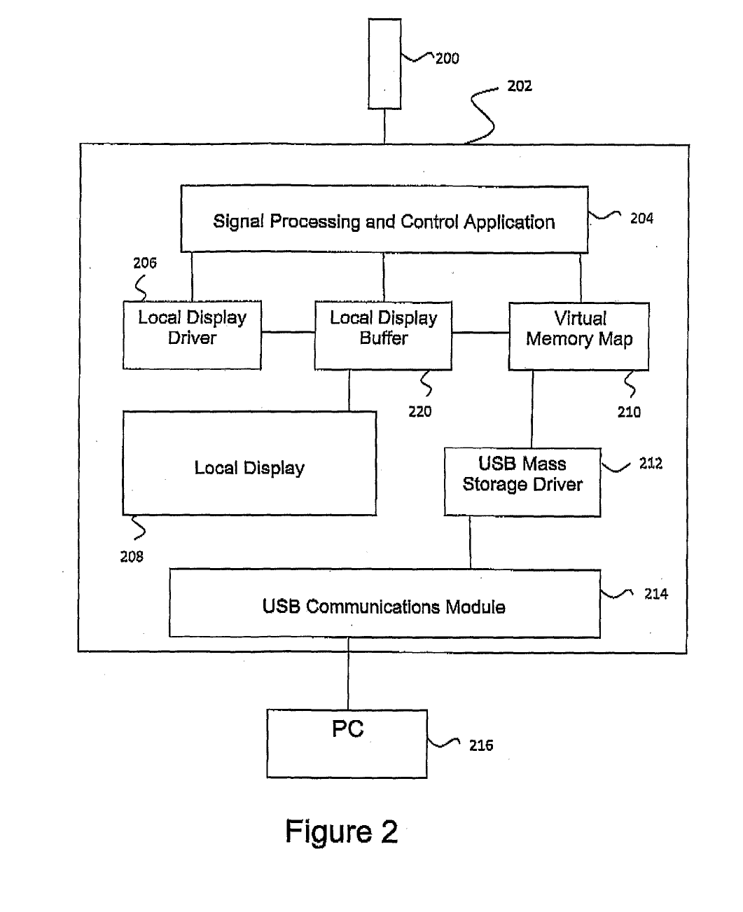

FIG. 2 shows a block diagram of a portable diagnostic device incorporating an embodiment of the invention.

Referring to FIG. 2, there is a probe unit 200 which is connected to a display and processing unit (DPU) 202. Signals from the probe unit 200 are received by the Signal Processing and Control Application 204. This application is specific to the functionality of the probe unit. It provides the basic functionality of receiving data from the probe unit, processing the data to produce diagnostic information, and formatting the information for display. This application also receives user input to control the probe and the display information.

The diagnostic information is provided to the Local Display Driver 206 for display on the Local Display Screen 208. The Signal Processing Application and the Local Display Driver co-operate to ensure that the diagnostic information is in a form suitable for display on the Local Display Screen 208.

In accordance with the invention, the display information is also made available for display an the Remote Display Unit, in this embodiment, a personal computer (PC) 216. The Signal Processing Application responds to user commands to achieve this.

The operating system of the DPU 202 includes a USB Mass Storage Driver 204. In the illustrated embodiment, it is a client driver that implements the USB mass storage device class. It is shipped as part of the Windows CE 5.0, which is the operating system employed in the preferred embodiment. Other operating systems would have analogous functionality.

The information to be displayed on the Local Display 208 is held in a memory block, the Local Display Buffer 220. The display information is shared with the Remote Display Unit by enabling at least part of the Local Display Buffer 220 to read by the Remote Display Unit.

The USB Mass Storage Driver 212 on the device maps the Local Display Buffer contents into it's own address space, the Virtual Memory Map 210, and can satisfy the requests received from the PC 216 with a simple memory copy operation.

The USB protocol allows for the insertion of additional commands, called vendor specific commands. A Vendor Specific command is added to read the Virtual memory Map. The Vendor Specific command implemented is similar to the standard USB Mass Storage read block command. The data to be read is described by specifying an offset within the buffer and a length of data to be read. Blocks up to 64 k-1 bytes can be read at a time with a 16 bit length counter. Larger blocks result in efficient transfers, and the device overhead is minimal.

The Vendor Specific command implemented on the device in the Mass Storage Driver 212 reads and validates the offset and length fields, and then performs a memory copy of the requested part of the Virtual Memory Map buffer into the reply, which will be of length requested in the command.

To use mass storage, a block device must be given, to the mass storage driver to use as the backing store for the storage. This may be a removable memory module or a RAM disk of minima) size may be created for this purpose.

Using a block device for Mass Storage requires that the mounted file system for that block device be dismounted whilst it is under control of the mast storage driver. This operation is automatically performed by the mass storage driver.

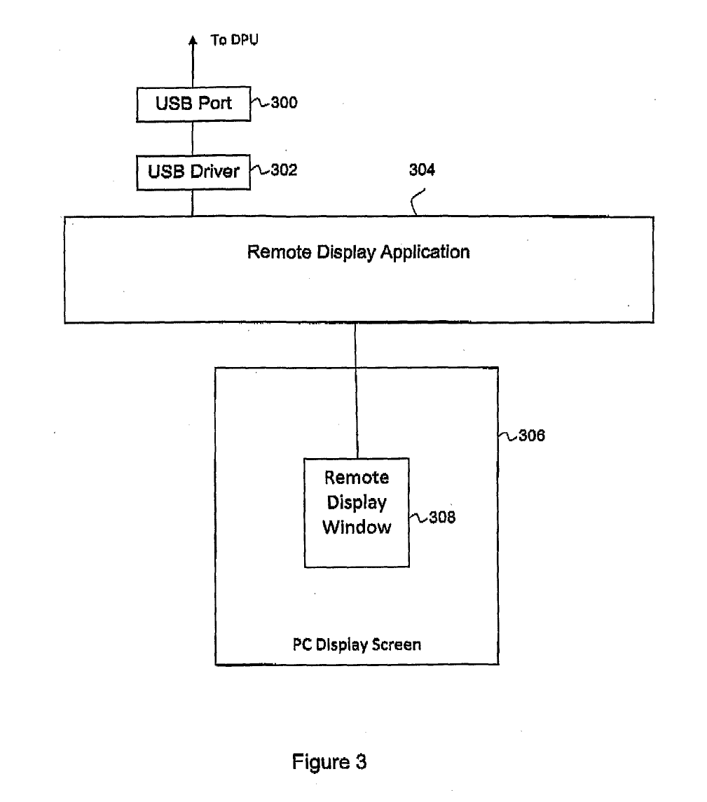

The Remote Display Unit 216 is a personal computer (PC) in the illustrated embodiments. A functional block diagram of a remote display unit is shown in FIG. 3.

There is a physical interface, in this case a USB connector 300. This could also be a Wi-Fi transducer, a Bluetooth transducer, or another suitable connector. Data is sent over this physical interface by an interface driver, in this case USB driver 302.

The USB data transfer protocol is implemented to communicate data over the physical interface.

An application runs on the PC to read the entire frame buffer of the device screen, at a desired frame rate. This is done by use of a Vendor Specific command of the USB protocol.

The frame buffer data read from the device is in the native pixel format of the device, in the embodiment 16-bit packed in 32 bit. This is unpacked on the PC, pixel by pixel. The PC has far greater resources to perform this conversion than the portable diagnostic device.

The PC can scale the output as desired, prior to display on the PC screen.

The Remote Display Unit, in this case a PC, runs the Remote Display Application 304. This application detects when a device is plugged in to the USB port 300 by use of functionality provided by the PC operating system. Specifically, the application registers for Volume notifications and uses Windows APIs to get the USB Vendor and Product IDs for connected USB volumes, if the Vendor and Product IDs match known values, the application has detected the connection of a portable diagnostic device.

Once a device is detected, the user is offered the ability to launch the Remote Display through a button. This button creates the Remote. Display Window 308, which is displayed on the PC display screen 306. The Window 308 may be resized, with the default size defaulting to full screen.

A timer is set for updates of the contents of the Remote Display Window. If the timer is set to 100 ms, this will give a frame rate of 10 frames per second. Each time a frame update is required, a new copy of the Virtual memory Map 210 is transferred from the connected device. The application keeps a file handle open to the volume, which is opened using the CreateFile API function. The window function DeviceIoControl is used to transfer a IOCTL_SCSI_PASS_THROUGH_DIRECT command to the USB driver 302 which contains the details of the Vendor Specific command to be sent to the device. On return, this function returns the status, and where successful, the data associated with the command.

The amount of data to be retrieved from the device for the screen of the embodiment is 4 bytes per pixel or 320*240*4=307200. As this exceeds the 16 bit length field, the screen is retrieved progressively, 32 k at time, using a Coop that increments the offset, and get each chunk of data. Note for 10 frames a second, this uses approx 3 MB/sec of data throughput.

Once the entire buffer is retrieved, each pixel is copied into a bitmap, converting the colour space in the process, ie taking the 18-bit colour value from the device, and converting it to a standard RGB colour value for windows. The window is then marked as invalid, and when a paint message arrives, the latest bitmap is painted to the screen, stretching the small size of the device screen (320×240) to the size of the window, preserving aspect ratio. If a failure occurs when reading the screen information, a check is made to see if the device is still connected, if not the window is closed. The window can also be closed by the user.

On the PC, the Vendor Specific commands require administrator privileges which is obtained using a service, a method known in the art.

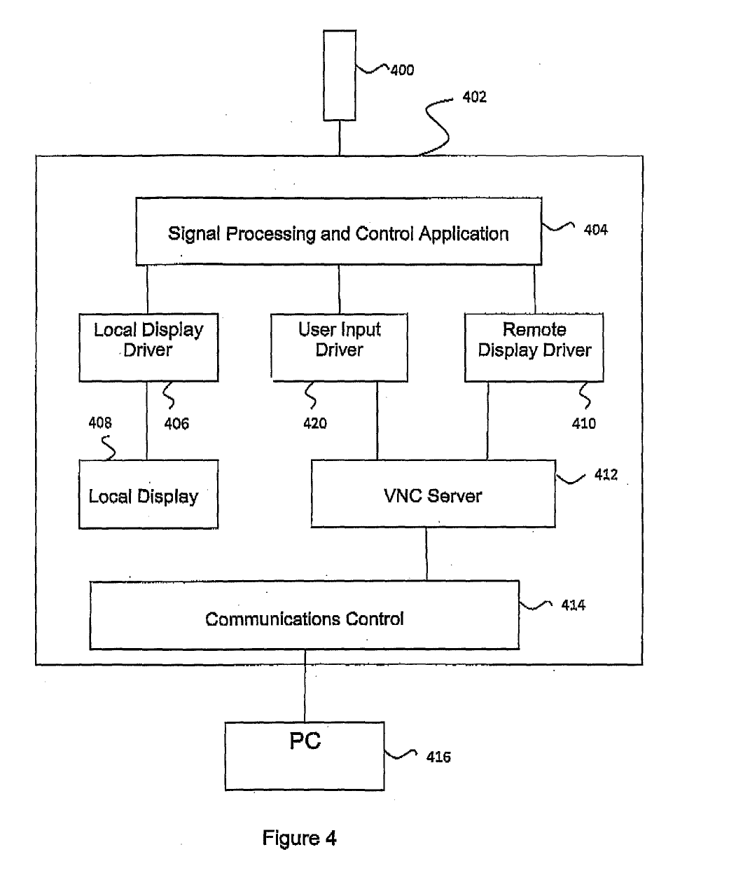

A block diagram of a further embodiment of the system is shown in FIG. 4. In this embodiment, corresponding applications are run on the portable diagnostic device and on the remote display unit. These applications provide communication of display information from the portable diagnostic device to the remote display unit and of control information from the remote display unit to the portable diagnostic device.

Referring to FIG. 4, there is a probe unit 400 which is connected to a display and processing unit (DPU) 402. Signals from the probe unit 400 are received by the Signal Processing and Control Application 404. This application is specific to the functionality of the probe unit. It provides the basic functionality of receiving data from the probe unit, processing the data to produce diagnostic information, and formatting the information for display. This application also receives user input to control the probe and the display information.

The diagnostic information is provided to the Local Display Driver 406 for display on the Local Display Screen 408. The Signal Processing Application and the Local Display Driver co-operate to ensure that the diagnostic information is in a form suitable for display on the Local Display Screen 408.

The diagnostic information is also provided to the Remote Display Driver 410 for display on the Remote Display Unit 418. The Signal Processing Application and the Remote Display Driver co-operate to ensure that the diagnostic information is in a form suitable for display on the Remote Display Unit screen.

The Remote Display Driver does not have direct access to the remote display. Having formatted the diagnostic information for display, the Remote Display Driver passes the display data to VNC server 412.

The VNC server is a software module which allows a human-machine interface to be shared with a remote device. In the case where only a local display driver was provided, this would mean that the display information provided to the remote display would be no different from the display information displayed locally by the DPU. This would be useful, but would not allow advantage to be taken of any greater or different display capabilities of the remote display.

The provision of a Remote Display Driver module 410, allows display information to be formatted into a format which uses the capabilities of the remote display. This includes different screen resolutions, and the display of additional information, made possible by the greater size of a remote display.

In an embodiment, the local and the remote display drivers may be combined. The local display may de-activated, or caused to display a static information message when the remote display is active.

The VNC server passes the display data to a communications channel driver 414, for transmission over a communications channel to the Remote Display Unit 416. The communication channel may be any convenient type of channel. It will be a channel of a type routinely provided on the device which forms the Remote Display Unit. Without limitation, these may be USB, Bluetooth, a wireless channel supporting the Ethernet protocol (WI-FI), or a wired Ethernet connection.

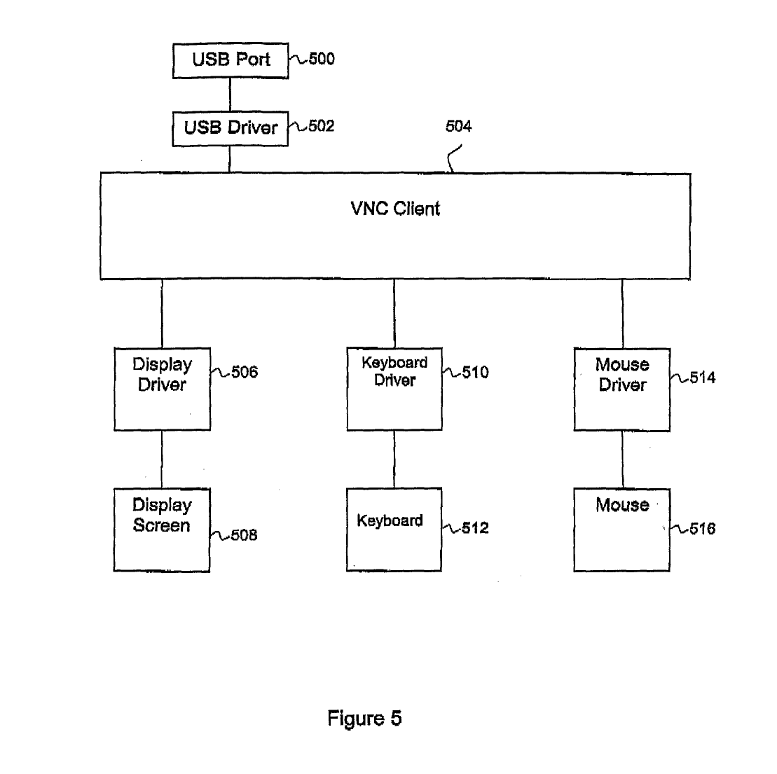

The Remote Display Unit 416 is a personal computer in the illustrated embodiments. A functional block diagram of a remote display unit is shown in FIG. 5.

There is a physical interface, in this case a USB connector 500. This could also be a Wi-Fi transducer, a Bluetooth transducer, or another suitable connector. Data is sent over this physical interface by an interface driver, in this case a USB driver 502.

A data transfer protocol is implemented to communicate data over the physical interface. Any convenient protocol may be chosen. In this embodiment it is the TCP/IP protocol.

Data is transferred to a VNC client program 504. This program passes frame buffer data received from the DPU to display driver 506 for display on remote display 508.

User control of the diagnostic device from the remote unit is also provided for. Keystrokes from keyboard 512 are passed to the VNC dent 504 via keyboard driver 510, to be passed to the DPU.

Movements of mouse 516 are likewise provided via mouse driver 514.

Referring again to FIG. 4, there is provided one or more User Input Drivers 420. This allows Signal processing and Control Application 404 to be controlled locally by the scrollwheel 5 and the pushbutton 6 and the touchscreen functionality of the local display 408. Where a local keyboard is provided, connected directly or wirelessly via Bluetooth or another protocol, this will also be controlled through drivers 420.

The personal computer which forms the Remote Display Unit 416 has a keyboard and pointing device, for example a mouse of touchpad. It does not have a scrollwheel or a dedicated pushbutton. It may have touchscreen functionality.

In order to allow the Signal Processing and Control Application to be controlled by user input devices, whether located locally or remotely, User Input Drivers 420 are provided for all input devices which may be located on the DPU or on the remote display device. User Input from the personal computer is received by the VNC server 412 and provided to the User Input Driver. This allows a user of the personal computer to control the scanning apparatus using a user interface that is the same as or different from that provided to a user of the DPU.

The user interface provided to the user of the remote display device is determined by the Signal Processing and Control Application and the user input and remote display drivers. In an embodiment where the remote device has other input means such as a scrollwheel, input for control of the diagnostic apparatus may be accepted from that input means.

VNC is a known method for remote control of a computer desktop. A VNC server runs on the DPU processor. A VNC client program runs on the remote display device, in this case a personal computer.

The server and the client negotiate an encoding scheme to use to communicate display and control information. The simplest scheme for transmitting display Information is one in which the server sends the contents of the display frame buffer to the client as a series of small rectangles. The server examines each rectangle of each subsequent frame in the frame buffer. Rectangles are resent only when they have changed since they were last transmitted.

In the case of medical scanning devices, such as ultrasound, only small regions of the screen are updated during a scan. This means that this encoding scheme requires relatively little bandwidth.

Any suitable method may be used to communicate display and control information between the DPU and a remote, display unit. The use of a standard, widely available interface means that no application-specific software is required at the remote unit. This means that the system can use a wide variety of computing devices as the remote unit without requiring additional development effort. Providing the interface to the remote unit with application specific software running on the remote device would require development and testing for many possible hardware and software combinations. In the system of the invention, the application specific software runs on the DPU. This dramatically reduces the development and testing effort required.

This embodiment has the advantage of providing remote control of the diagnostic device from the remote display unit. Control actions applied at the remote display unit such as mouse movements and keyboard input may be transmitted to the diagnostic device. Running the VNC applications uses greater processing resources at the remote display unit and particularly at the portable diagnostic device, where such resources are less likely to be readily available.

An alternative embodiment uses X Windows to provide the remote display functionality.

The remote device, such as a personal computer, PDA or mobile phone runs an X server program. The DPU runs an X client program. The X server running on the remote display device communicates with the X client running on the DPU. The client sends requests to the server to display graphical data which the server decodes and sends to the remote display screen for display to the user. The server sends back user input from keyboard, mouse, touchscreen or other user input device to the client, to be passed to the Signal Processing and Control Application to implement user control.

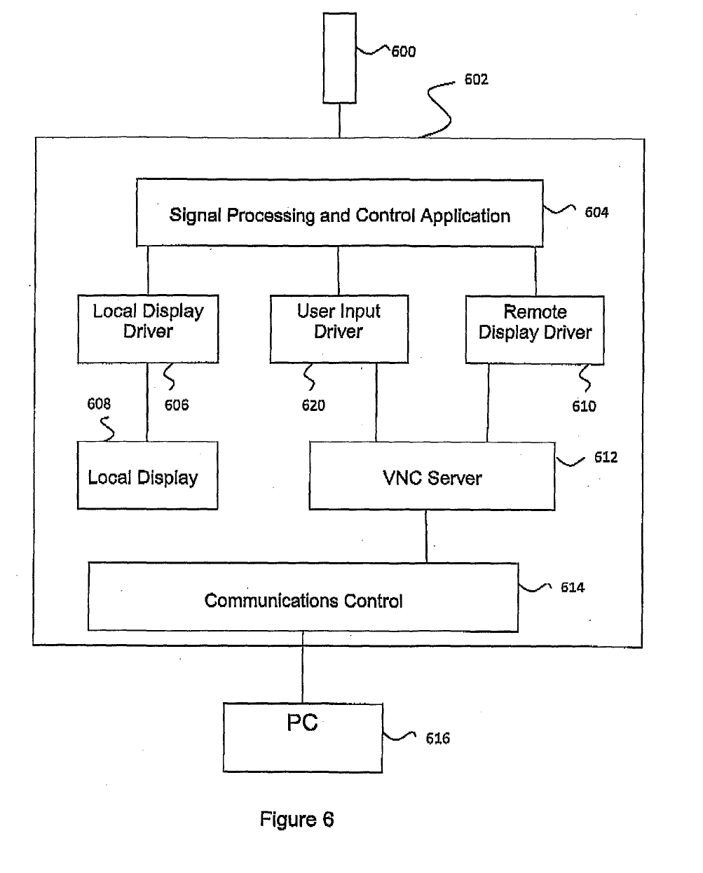

FIG. 6 shows a further embodiment in which no local display is provided. The probe unit 61 includes all of the functionality of the DPU of the previously described embodiments, except that no display is provided. A connection cable 62 is provided to link the probe unit to a remote display unit 63. This remote display unit may be any device with a general computing function. Without limitation, it may be a computer a PDA or a mobile phone.

Alternatively or in addition, the link may be provided by a wireless Link 64. This wireless link may be provided by Wi-Fi, Bluetooth, or any other convenient protocol.

The implementation of the system is as described in the description of the embodiment of FIG. 4 and FIG. 5, with the exception that no local display or local display driver are provided.

In this embodiment, it is necessary for the probe unit to make contact with a remote display unit before it can begin to function. Accordingly, it attempts to make such contact on start-up, and continues such attempts periodically until successful.

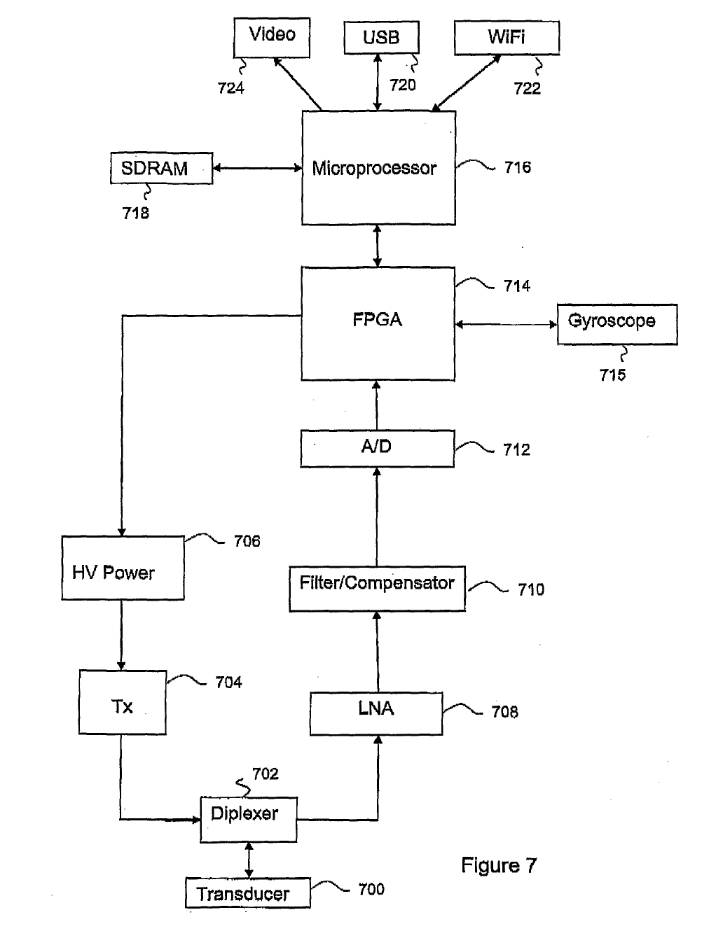

A block diagram of a probe unit of the embodiment of FIG. 6 is shown in FIG. 7. In this embodiment, the probe unit is an ultrasound scanning unit.

There is a transducer 700 which sends ultrasound pulses into the body of a patient to be scanned. These pulses are reflected from features of the body. The reflections are then received by the transducer 700, and converted to electrical signals.

A diplexer 702 connects the transducer to a high voltage electrical pulse transmitter 704 to receive high voltage pulses, powered by the high voltage power supply 705. These high voltage pulses are converted to ultrasonic pulses transmitted into the body to be scanned.

When the reflected signals are to be received, the diplexer 702 connects the transducer to electrical signet receiving circuitry. There is a low noise amplifier 708 which amplifies the small electrical signal from the transducer, whilst introducing as little noise as possible.

The amplified signal is then passed to a time gain amplifier and a low pass filter.

The time gain amplifier applies a ramped amplification to the signal to adjust for the attenuation of ultrasound by body tissue. This attenuation means that echoes from similar features deeper in the body are weaker than those from nearer the surface. Applying a carefully selected ramped amplification compensates for this effect.

The low pass filter removes high frequency components of the signal to prevent aliasing by the analogue to digital converter 712 to which the signal is now passed.

The analogue to digital converter converts the received signal to digital data which is passed to a field programmable gate array (FPGA) 714.

There is also provided a position and orientation sensor, which provided information about the relative position and/or orientation of the probe unit. Any suitable sensor system may be employed. The sensor may, without restriction, operate of inertial, electro-magnetic or optical principles. It may detect linear or angular movement in any number of degrees of freedom.

In the described embodiment, the position and orientation sensor is an inertial sensor in the form of gyroscope 715. The angular position information from the gyroscope is passed to the FPGA 714.

The FPGA implements a signal processing function which combines the received data and formats it as ultrasound scanlines. These scanlines, including echo intensity and probe orientation information are passed to the microprocessor 716 which has storage provided by SDRAM 718.

The microprocessor runs a Signal Processing and Control Application, which formats the received scanlines as an ultrasound image.

This application also modifies the image under user or program control, and adds user data such as callipers, tags and other display information.

This application also controls the high voltage transmitter 704, allowing user control of the ultrasound transmission.

The microprocessor controls all external communications from the probe unit. It controls either or both of a USB interface 720 and a Wi-Fi interface 722. It may also have a direct video output 724.

The microprocessor runs a compact operating system such as Linux or Windows CE. This operating system runs a remote display program, in this embodiment a VNC server. It might also be an X windows client or another such program. It also provides user input and output drivers.

No local display is provided. Accordingly the VNC server provides an interface to a remote display and control device (not shown). This is a device with a general purpose computing function such as a computer, a PDA or a mobile phone.

Data for display is formatted into a screen buffer by the microprocessor 716. The screen buffer is transmitted to the remote display device as described for the embodiment of FIG. 4 and FIG. 5. Control signals from the remote display device are transmitted to the microprocessor by the VNC server. Such control signal may be from keyboard, scrollwheel, touchscreen or any other appropriate control mechanism.

In a further embodiment, the remote display unit may be a display terminal only, without the necessary processing functionality to run a VNC client. In this embodiment, the DPU 1 includes circuitry enabling the DPU to drive an external display directly.

Although the invention has been herein shown and described in what is conceived to be the most practical and preferred embodiment, it is recognised that departures can be made within the scope of the invention, which is not to be limited to the details described herein but is to be accorded the full scope of the appended claims so as to embrace any and all equivalent devices and apparatus.

1-19. (canceled)

20. A hand held medical diagnostic apparatus including a processing unit;
a probe which produces as an output medical diagnostic data;
the processing unit receiving said medical diagnostic data and processing the data to produce display information for display to a user;
a communications channel which transmits data between the processing unit and a remote display unit located remotely from the probe and from the processing unit;
wherein the processing unit sends the display information via the communications channel to the remote display unit and the remote display unit displays the display information on a screen.

21. The apparatus of claim 20 wherein the communications channel is a USB communications channel, and the display information is transmitted by use of the Mass Storage function of the USB protocol including the Vendor Specific command functionality of the USB protocol.

22. The hand held medical diagnostic apparatus of claim 20 wherein the probe unit and the processing unit are located in a common housing.

23. The hand held medical diagnostic apparatus of claim 20 wherein the probe unit and the processing unit are located in separate housings.

24. The apparatus of claim 20 wherein the remote display unit is selected from a PDA, a personal computer and a mobile telephone.

25. The apparatus of claim 20 wherein the processing unit housing includes a local display screen and the processing unit displays the display information on the local display screen.

26. The apparatus of claim 25 wherein the display specifications of the remote display unit screen are significantly different from the display specifications of the local display screen and the display information displayed on each of the screens by the processing unit in response to the medical diagnostic data is substantially different.

27. The apparatus of claim 23 wherein the processing unit housing does not include a local display screen and the processing unit is adapted to display the display information only on the remote display unit screen.

28. The apparatus of claim 20 wherein the medical diagnostic apparatus is a hand held diagnostic ultrasound and the probe unit is an ultrasound scanning probe.

29. The apparatus of claim 20 wherein the physical implementation of the communications channel is selected from a USB connection, a Bluetooth connection and a wireless Ethernet connection.

30. The apparatus of claim 20 wherein the communications channel utilises the TCP/IP communications protocol.

31. A hand held medical diagnostic apparatus including a processing unit;
the processing unit having user interface controls by which a user controls the operation of the
a probe which produces as an output medical diagnostic data;
the processing unit receiving said medical diagnostic data and processing the data to produce display information for display to a user;
a communications channel which transmits data between the processing unit and a remote display unit located remotely from the probe and from the processing unit, the remote display unit being equipped with at least one of the types of user interface control provided at the processing unit;
wherein the processing unit sends the display information via the communications channel to the remote display unit, the remote display unit displays the display information on a screen, the remote display unit further communicates to the processing unit data describing any manipulation by a user of the user interface controls on the remote display unit, the processing unit processing that data to implement remote control of the medical diagnostic apparatus by said user of the remote display unit.

32. The apparatus of claim 31 wherein the user interface controls which may be operated at the remote display init include a scrollwheel.

33. The apparatus of claim 31 wherein the user interface controls which may be operated at the remote display init include a touch sensitive screen.

34. The apparatus of claim 20 wherein the processing unit and the remote display unit run complementary remote control and display software applications.

35. The apparatus of claim 34 wherein the remote control and display software applications are the VNC suite of programs.
